(19) United States
(12) Patent Application Publication
(10) Pub. No.: US 2024/0281396 A1
(43) Pub. Date: Aug. 22, 2024

(54) COMMUNICATION ADAPTER AND METHOD FOR TRANSFERRING DATA

(71) Applicant: BIOTRONIK SE & Co. KG, Berlin (DE)

(72) Inventors: Thomas DOERR, Berlin (DE); Ulrich FEESE, Berlin (DE)

(73) Assignee: BIOTRONIK SE & Co. KG, Berlin (DE)

(21) Appl. No.: 18/570,162

(22) PCT Filed: Jun. 9, 2022
(86) PCT No.: PCT/EP2022/065668

§ 371 Date: Dec. 14, 2023

(30) Foreign Application Priority Data
Jul. 16, 2021 (EP) 21186003.6

(51) Int. Cl. G06F 13/38; A61N 1/372; A61N 1/378; G06F 13/42; H04M 1/02

(52) U.S. Cl. CPC G06F 13/382; A61N 1/37229; A61N 1/37254; A61N 1/3787; G06F 13/4282; H04M 1/0274; G06F 2213/0042

(57) ABSTRACT

A communication adapter and a computer implemented method for use with an implantable medical device, in particular a pacemaker, a defibrillator and/or a neuro-stimulator, for transferring data between the implantable medical device and a mobile device, in particular a smartphone or tablet computer. In addition, the invention relates to a protective case, a cable and a connector each comprising the communication adapter.

### CROSS-REFERENCE TO RELATED APPLICATIONS

This application is the United States National Phase under 35 U.S.C. § 371 of PCT International Patent Application No. PCT/EP2022/065668, filed on Jun. 9, 2022, which claims the benefit of European Patent Application No. 21186003.6, filed on Jul. 16, 2021, the disclosures of which are hereby incorporated by reference herein in their entireties.

### TECHNICAL FIELD

The present invention relates to a communication adapter for use with an implantable medical device, in particular a pacemaker, a defibrillator, a cardiac monitor and/or a neuro-stimulator, for transferring data between the implantable medical device and a mobile device, in particular a smartphone or tablet computer.

Furthermore, the present invention relates to a protective case, a cable and a connector each comprising the communication adapter.

In addition, the present invention relates to a computer implemented method for transferring data between an implantable medical device, in particular a pacemaker, a defibrillator, a cardiac monitor and/or a neuro-stimulator, and a mobile device, in particular a smartphone or tablet computer, by means of a communication adapter.

### BACKGROUND

European Publication No. 1 762 955 A1 discloses a communication adapter for use with a portable ambulatory medical or therapeutic device, in particular a device for the diagnosis or treatment of a glucose metabolism disorder, for transferring data between the medical or therapeutic device and a computer for displaying operating parameters or measurement data of the device and/or for operating the device, wherein the medical or therapeutic device comprises a device processor for controlling the device and a device adapter interface for communication of the device processor with the communication adapter, and wherein the communication adapter comprises an adapter processor for controlling the communication adapter, an adapter device interface for communication of the communication adapter with the device, an adapter computer interface for communication of the adapter processor with a computer interface of the computer and a device driver with associated transmission protocol.

Implant systems that are able to communicate with a mobile device such as a smart phone are usually equipped with a Bluetooth Low Energy interface.

Disadvantages of a Bluetooth Low Energy interface in implants are, on the one hand, an increased power consumption of this telemetry function especially if the connection is established frequently and, on the other hand, the changing standardizations and possible discontinuation of the Bluetooth Low Energy transmission protocol on the smart-phone side over time.

There is thus a risk that no suitable smart-phone that can communicate with the Bluetooth Low Energy interface of the implant will be available throughout the operating life of the implant which is typically 15 years.

The present disclosure is directed toward overcoming one or more of the above-mentioned problems, though not necessarily limited to embodiments that do.

### SUMMARY

It is therefore an object of the present invention to provide an improved communication adapter for use with an implantable medical device that offers low energy consumption and that can communicate with the interface of the implant throughout its operating life.

At least the object is solved by a communication adapter for use with an implantable medical device, in particular a pacemaker, a defibrillator, a cardiac monitor and/or a neuro-stimulator, for transferring data between the implantable medical device and a mobile device, in particular a smartphone or tablet computer having the features of claim 1.

At least the object is furthermore solved by a protective case having the features of claim 7, a cable having the features of claim 8 and a connector having the features of claim 9.

In addition, at least the object is solved by a computer implemented method for transferring data between an implantable medical device, in particular a pacemaker, a defibrillator, a cardiac monitor and/or a neuro-stimulator, and a mobile device, in particular a smartphone or tablet computer, by means of a communication adapter having the features of claim 11.

Moreover, at least the object is solved by a computer program having the features of claim 12 and a computer-readable data carrier having the features of claim 13.

Further developments and advantageous embodiments are defined in the dependent claims.

The present invention provides a communication adapter for use with an implantable medical device, in particular a pacemaker, a defibrillator, a cardiac monitor and/or a neuro-stimulator, for transferring data between the implantable medical device and a mobile device, in particular a smartphone or tablet computer.

The communication adapter comprises a MICS telemetry interface for data transfer between the communication adapter and the implantable medical device.

The communication adapter further comprises a communication and power supply interface for data transfer between the communication adapter and the mobile device and a connector for connecting to the mobile device, wherein the communication adapter is powered via the communication and power supply interface and wherein the communication adapter is configured to transfer data sent via the MICS telemetry interface by the implantable medical device to the mobile device via the communication and power supply interface and to transfer data sent via the communication and power supply interface by the mobile device to the implantable medical device via the MICS telemetry interface.

Furthermore, the present invention provides a protective case for a mobile device comprising a frame, in particular made from an elastic material, mountable around an edge of the mobile device, in particular a smartphone or tablet computer; and the communication adapter of the present invention.

In addition, the present invention provides a cable comprising a connector, in particular a USB type-A, USB-OTG, USB-C, Mini-USB, Micro-USB, Lightning or Thunderbolt connector; and the communication adapter of the present invention.

The present invention further provides a computer implemented method for transferring data between an implantable medical device, in particular a pacemaker, a defibrillator, a cardiac monitor and/or a neuro-stimulator, and a mobile device, in particular a smartphone or tablet computer, by means of a communication adapter.

The method comprises providing a MICS telemetry interface for data transfer between the communication adapter and the implantable medical device.

The method further comprises providing a communication and power supply interface for data transfer between the communication adapter and the mobile device, wherein the communication adapter transfers data sent via the MICS telemetry interface by the implantable medical device to the mobile device via the communication and power supply interface and transfers data sent via the communication and power supply interface by the mobile device to the implantable medical device via the MICS telemetry interface.

An idea of the present invention is to provide a communication adapter that converts a communication standard of a mobile device to a communication standard of an implantable medical device, namely a MICS band telemetry. Using MICS band telemetry, the implantable medical device can thus communicate easily and inexpensively with a mobile device such as a smart phone throughout the operating life of the implant which is typically 15 years.

An example of a purely therapeutic implant/implantable medical device is, e.g., a stimulator/electrode for deep brain stimulation (e.g., Parkinson's therapy or therapy of depression). The therapy consists of the delivery of pulse trains (120) without collecting diagnostic data from the stimulator.

An example of a purely diagnostic implant is, e.g., a cardiac rhythm monitor. The diagnostic function consists of continuous recording of the patient's ECG and automatic evaluation of abnormalities of the heart rhythm. If such are detected, an ECG recording is stored and typically automatically transmitted to a remote monitoring system.

An example of an implant with therapeutic and diagnostic functions is, e.g., a cardiac pacemaker. The pacemaker is typically implanted subcutaneously in the upper right thoracic region and the electrode is placed in the patient's heart via a large vein. The therapeutic function consists of delivering stimulation pulses to trigger a cardiac action, provided there is no spontaneous cardiac action in the patient. The diagnostic function consists, for example, in the continuous recording of the patient's ECG and automatic evaluation of abnormalities of the heart rhythm. If such are detected, an ECG recording is stored and typically automatically transmitted to a remote monitoring system.

According to an aspect of the present invention, the communication and power supply interface for data transfer between the communication adapter and the mobile device, can be realized using USB On-The-Go (USB-OTG).

USB-OTG is a specification, that allows USB devices, such as tablets or smartphones, to act as a host, allowing other USB devices, such as USB drives, digital cameras or keyboards, to be attached to them. A smartphone may read from removable media as the host, but present itself as a (USB Mass Storage) device when connected to a host computer. USB-OTG introduces the concept of an appliance performing both host and device roles—whenever two USB appliances are connected and one of them is a USB-OTG device, they establish a communication link. The appliance controlling the link is called the host, while the other is called the device or peripheral.

When a device is plugged into a USB bus, the master device, or host, sets up communications with the device and handles service provisioning (the host's software enables or does the needed data-handling). That allows the communication and power supply interface (device/peripheral) to be greatly simplified compared to the host (tablet or smartphone).

According to an aspect of the present invention, the communication adapter is configured to connect the mobile device with a further device, in particular a charging device, a computing device and/or a data storage device, by a USB connector.

Hence, the communication adapter is configured such that the port remains usable after the adapter is attached by replicating the communication and power supply port of the mobile device. The user can then connect his charging cable to the smartphone, for example. The communication and power supply port of the smartphone is thus not blocked. The connection socket of the smartphone is replicated.

According to a further aspect of the present invention, the communication adapter is integratable into a protective case of the mobile device, into a cable or into a connector. This way, the communication and power supply port of the mobile device is usable for other functions while at the same time providing MICS telemetry functionality in order to connect the mobile device to the implantable medical device.

According to a further aspect of the present invention, the MICS telemetry interface comprises a MICS-band-radio module comprising an encryption unit, in particular a hardware- and/or software-based encryption unit configured to encrypt a communication between the MICS telemetry interface and the implantable medical device. The communication between the mobile device and the implantable medical device can thus be rendered more secure.

According to a further aspect of the present invention, the communication adapter comprises or is connected to at least a first MICS-band antenna and a second MICS-band antenna. This provides advantage of antenna diversity.

According to a further aspect of the present invention, the communication adapter comprises an authentication unit, in particular a hardware- and/or software-based authentication unit, configured to authenticate a user of the mobile device in order to access patient-related data stored in a data storage unit of the communication adapter and/or the implantable medical device. By authenticating the user of the mobile device, access to the implantable medical device can be restricted to only authorized users. This provides an additional layer of security.

According to a further aspect of the present invention, the communication adapter is adapted such that the energy source is replaceable or rechargeable. This enables ease of use and facilitates replacement of the energy source when necessary.

The herein described features of the communication adapter for use with an implantable medical device are also disclosed for the computer implemented method for transferring data between an implantable medical device and a mobile device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a schematic view of a communication adapter for use with an implantable medical device according to a first embodiment of the present invention;

FIG. 2 shows a schematic view of the mobile device arranged within a protective cover according to the first embodiment of the present invention:

FIG. 3 shows a schematic view of a communication adapter for use with an implantable medical device according to a second embodiment of the present invention:

FIG. 4 shows a schematic view of a communication adapter for use with an implantable medical device according to a third embodiment of the present invention:

FIG. 5 shows a schematic view of the communication adapter of FIG. 4 for use with an implantable medical device according to the third embodiment of the present invention; and

FIG. 6 shows a flowchart of a computer implemented method for transferring data between an implantable medical device and a mobile device according to the first to third embodiments of the present invention.

### DETAILED DESCRIPTION

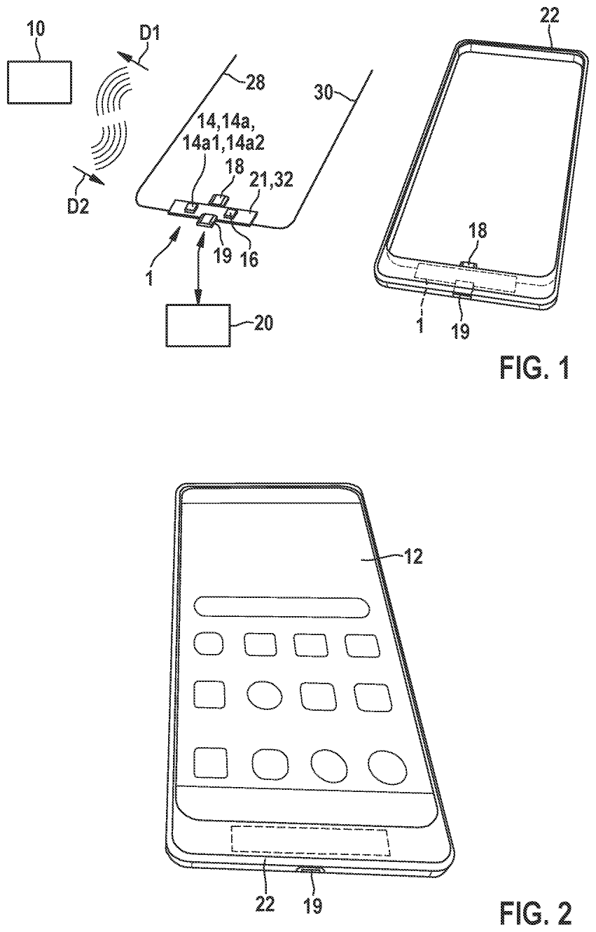

The communication adapter 1 of FIG. 1 for use with an implantable medical device 10, in particular a pacemaker, a defibrillator, a cardiac monitor and/or a neuro-stimulator, for transferring data D1, D2 between the implantable medical device 10 and a mobile device 12, in particular a smartphone or tablet computer, comprises a MICS telemetry interface 14 for data transfer between the communication adapter 1 and the implantable medical device 10.

The communication adapter 1 further comprises a communication and power supply interface 16, in particular a USB-OTG, USB-C, Lightning or Thunderbolt interface, for data transfer between the communication adapter 1 and the mobile device 12 and for power supply from the mobile device 12 to the communication adapter 1, a connector 18 for connecting to the mobile device 12 and a USB connector 19, in particular a USB port identical to the USB port featured for USB-OTG of the mobile device 12 such that the USB port, in particular a USB-C port, of the mobile device 12 is effectively replicated. In doing so, it is possible to connect, e.g., a USB cable through the USB connector 19 to the mobile device 12 for charging and/or data transfer. Alternatively, the port of the mobile device 12 can be a Lightning or Thunderbolt port.

The communication adapter 1 is powered via the communication and power supply interface 16. Furthermore, the communication adapter 1 is configured to transfer data D1 sent via the MICS telemetry interface 14 by the implantable medical device 10 to the mobile device 12 via the communication and power supply interface 16 and to transfer data D2 sent via the communication and power supply interface 16 by the mobile device 12 to the implantable medical device 10 via the MICS telemetry interface 14.

The communication adapter 1 is configured to connect the mobile device 12 with a further device 20, in particular a charging device, a computing device and/or a data storage device, by a USB, USB-OTG, Lightning or Thunderbolt connector.

The communication adapter 1 is integratable into a protective case 22 of the mobile device 12, into a USB-cable 122 or into a connector 222. The MICS telemetry interface 14 comprises a MICS-band-radio module 14a comprising an encryption unit 14a1 in particular a hardware- and/or software-based encryption unit 14a1, configured to encrypt a communication between the MICS telemetry interface 14 and the implantable medical device 10.

The communication adapter 1 comprises or is connected to at least a first MICS-band antenna 28 and a second MICS-band antenna 30. The first MICS-band antenna 28 and the second MICS-band antenna 30 are adapted to a form of the protective cover 22 and are arranged to extend out from both sides of a circuit board 21 of the convocation adapter and continue along the vertical, i.e., longer sides of the protective cover 22.

The communication adapter 1 comprises an authentication unit 14a2, in particular a hardware- and/or software-based authentication unit 14a2, configured to authenticate a user of the mobile device 12 in order to access patient-related data stored in a data storage unit 32 of the communication adapter 1 and/or the implantable medical device 10.

The communication adapter is integratable into a protective case 22 for a mobile device 12. The protective case 22 comprises a frame, in particular made from an elastic material, mountable around an edge of the mobile device 12, in particular a smartphone or tablet computer; and the communication adapter 1.

FIG. 2 shows a schematic view of the mobile device 12 arranged within a protective cover 22 according to the first embodiment of the present invention. The protective cover 22 has greater dimensions at the bottom of the mobile device such that the communication adapter can fit between a lower edge of the mobile device 12 and an inner edge of the protective cover 22.

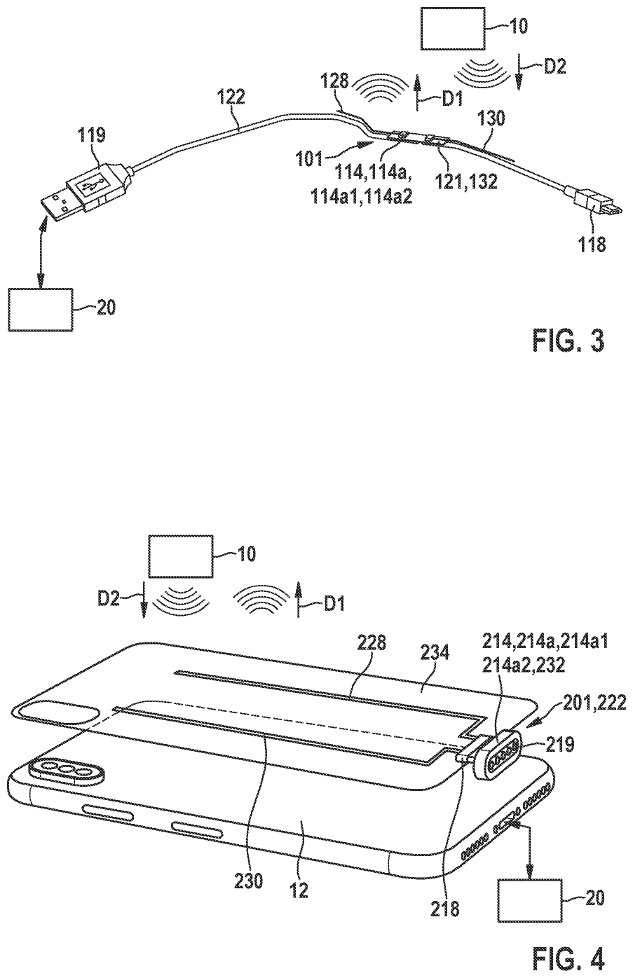

FIG. 3 shows a schematic view of a communication adapter 101 for use with an implantable medical device 10 according to a second embodiment of the present invention. The communication adapter is integrated into a USB-cable 122.

The USB-cable 122 comprises the communication adapter 101, the connector 118 for connecting to the mobile device 12 and a USB connector 119, in particular a USB type-A connector. The connector 118 can, e.g., be a USB-C connector or a Lightning connector for compatibility with Apple products or any other comparable connector who provide power supply for the adapter and support data exchange between the adapter and the mobile device.

The communication adapter 101 of FIG. 3 is capable of transferring data D1, D2 between the implantable medical device 10 and a mobile device 12 and comprises a MICS telemetry interface 114 for data transfer between the communication adapter 101 and the implantable medical device 10.

The communication adapter 101 further comprises a communication and power supply interface 16 for data transfer between the communication adapter 101 and the mobile device 12 and a connector 118 for connecting to the mobile device 12. The connector 118 is linked to the communication adapter 101 by means of the USB-cable 122.

In addition, the USB connector 119, in particular the USB type-A connector, is connected to communication adapter 101 by means of the USB-cable 122. That way, it is possible to connect the USB-cable 122 to a further device 20, in particular a charging device, a computing device and/or a data storage device.

The communication adapter 101 is powered via the communication and power supply interface 16. Furthermore, the communication adapter 101 is configured to transfer data D1 sent via the MICS telemetry interface 114 by the implantable medical device 10 to the mobile device 12 via the communication and power supply interface 16 and to transfer data D2 sent via the communication and power supply interface 16 by the mobile device 12 to the implantable medical device 10 via the MICS telemetry interface 114.

The MICS telemetry interface 114 comprises a MICS-band-radio module 114a comprising an encryption unit 114a1 in particular a hardware- and/or software-based encryption unit 114a1, configured to encrypt a communication between the MICS telemetry interface 114 and the implantable medical device 10.

The communication adapter 101 comprises or is connected to at least a first MICS-band antenna 128 and a second MICS-band antenna 130. The first MICS-band antenna 128 and the second MICS-band antenna 130 arranged to extend along the USB-cable 122.

The communication adapter 101 comprises an authentication unit 114a2, in particular a hardware- and/or software-based authentication unit 114a2, configured to authenticate a user of the mobile device 12 in order to access patient-related data stored in a data storage unit 132 of the communication adapter 101 and/or the implantable medical device 10.

FIG. 4 shows a schematic view of a communication adapter 201 for use with an implantable medical device 10 according to a third embodiment of the present invention. The communication adapter 201 is integrated into a connector 222.

The connector 222 comprises two USB connectors, the first one of which is adapted to plug into the mobile device 12 and the second of which provides an additional USB connector 219 identical to a USB port of the mobile device 12 such that the USB port of the mobile device is effectively replicated. In doing so, it is possible to connect, e.g., a USB cable through the USB connector 19 to the mobile device 12 for charging and/or data transfer.

The communication adapter 201 of FIG. 4 is capable of transferring data D1, D2 between the implantable medical device 10 and a mobile device 12 and comprises a MICS telemetry interface 214 for data transfer between the communication adapter 201 and the implantable medical device 10. The communication adapter 201 further comprises a communication and power supply interface 16 for data transfer between the communication adapter 201 and the mobile device 12.

The communication adapter 201 is powered via the communication and power supply interface 16, for example using USB-OTG. Furthermore, the communication adapter 201 is configured to transfer data D1 sent via the MICS telemetry interface 214 by the implantable medical device 10 to the mobile device 12 via the communication and power supply interface 16, for example using USB-OTG and to transfer data D2 sent via the communication and power supply interface 16 by the mobile device 12 to the implantable medical device 10 via the MICS telemetry interface 214.

The MICS telemetry interface 214 comprises a MICS-band-radio module 214a comprising an encryption unit 214a1 in particular a hardware- and/or software-based encryption unit 214a1, configured to encrypt a communication between the MICS telemetry interface 214 and the implantable medical device 10.

The communication adapter 201 is connected to at least a first MICS-band antenna 228 and a second MICS-band antenna 230, wherein a first MICS-band antenna 228 and a second MICS-band antenna 230 are arranged on a surface of an adhesive film 234, said adhesive film 234 being attachable to the mobile device 12.

The communication adapter 201 further comprises an authentication unit 214a2, in particular a hardware- and/or software-based authentication unit 214a2, configured to authenticate a user of the mobile device 12 in order to access patient-related data stored in a data storage unit 232 of the communication adapter 201 and/or the implantable medical device 10.

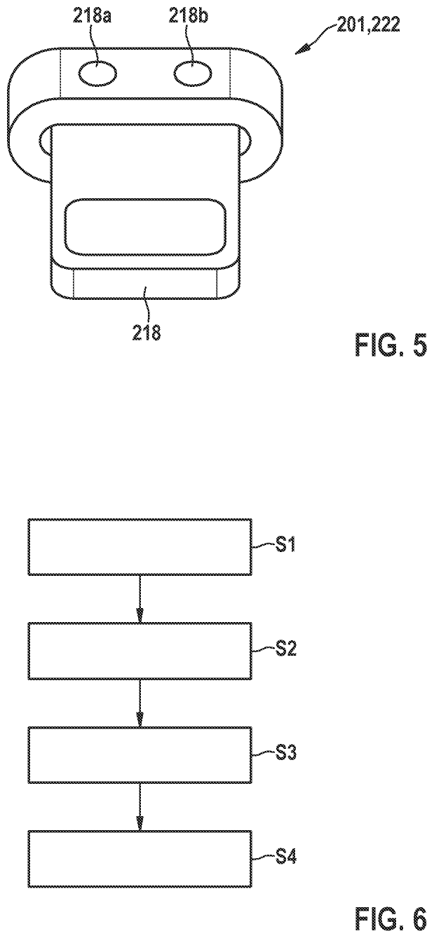

FIG. 5 shows a schematic view of the communication adapter for use with an implantable medical device according to the third embodiment of the present invention.

The connector 222 comprises the communication adapter 201. Furthermore, the connector 222 comprises a first antenna connection 218a to which the first MICS-band antenna 228 is connectable and a second antenna connection 218b, to which the second MICS-band antenna 230 is connectable.

FIG. 6 shows a flowchart of a computer implemented method for transferring data between an implantable medical device 10, in particular a pacemaker, a defibrillator, a cardiac monitor and/or a neuro-stimulator, and a mobile device 12, in particular a smartphone or tablet computer, by means of a communication adapter 1, 101, 201 according to the first to third embodiments of the present invention.

The method comprises providing S1 a MICS telemetry interface 14, 114, 214 for data transfer between the communication adapter 1, 101, 201 and the implantable medical device 10.

Furthermore, the method comprises providing S2 a communication and power supply interface 16, for data transfer between the communication adapter 1, 101, 201 and the mobile device 12, wherein the communication adapter 1, 101, 201 transfers data S3 sent via the MICS telemetry interface 14, 114, 214 by the implantable medical device 10 to the mobile device 12 via the communication and power supply interface 16 and transfers data S4 sent via the communication and power supply interface 16 by the mobile device 12 to the implantable medical device 10 via the MICS telemetry interface 14, 114, 214.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1, 101, 201 communication adapter
10 implantable medical device
12 mobile device
14, 114, 214 MICS telemetry interface
14a, 114a, 214a MICS-band-radio module
14a1, 114a1, 214a1 encryption unit
14a2, 114a2, 214a2 authentication unit
16 communication and power supply interface
18, 118, 218, 222 connector
19, 119, 219 USB connector
20 further device
21 circuit board
22 protective case
218a first antenna connection
218b second antenna connection
122 USB-cable
28, 128, 228 first MICS-band antenna
30, 130, 230 second MICS-band antenna
32, 132, 232 data storage unit
234 adhesive film
D1, D2 data
S1-S4 method steps

1. Communication adapter for use with an implantable medical device, in particular a pacemaker, a defibrillator, a cardiac monitor and/or a neuro-stimulator, for transferring data between the implantable medical device and a mobile device, in particular a smartphone or tablet computer, comprising:
a MICS telemetry interface for data transfer between the communication adapter and the implantable medical device;
a communication and power supply interface, in particular a USB-OTG, USB-C, Lightning or Thunderbolt interface, for data transfer between the communication adapter and the mobile device and for power supply from the mobile device to the communication adapter; and
a connector, in particular a USB-C, Lightning or Thunderbolt connector, for connecting to the mobile device, wherein the communication adapter is powered via the communication and power supply interface, and wherein the communication adapter is configured to transfer data sent via the MICS telemetry interface, by the implantable medical device to the mobile device via the communication and power supply interface and to transfer data sent via the communication and power supply interface by the mobile device to the implantable medical device via the MICS telemetry interface.

2. Communication adapter of claim 1, wherein the communication adapter is configured to connect the mobile device with a further device, in particular a charging device, a computing device and/or a data storage device, by a USB connector.

3. Communication adapter of claim 1, wherein the communication adapter is integratable into a protective case of the mobile device, into a USB-cable or into a connector.

4. Communication adapter of claim 1, wherein the MICS telemetry interface comprises a MICS-band-radio module comprising an encryption unit, in particular a hardware- and/or software-based encryption unit, configured to encrypt a communication between the MICS telemetry interface and the implantable medical device.

5. Communication adapter of claim 1, wherein the communication adapter comprises or is connected to at least a first MICS-band antenna and a second MICS-band antenna.

6. Communication adapter of claim 1, wherein the communication adapter comprises an authentication unit, in particular a hardware- and/or software-based authentication unit, configured to authenticate a user of the mobile device in order to access patient-related data stored in a data storage unit of the communication adapter and/or the implantable medical device.

7. Protective case for a mobile device comprising a frame, in particular made from an elastic material, mountable around an edge of the mobile device, in particular a smartphone or tablet computer; and the communication adapter of claim 1.

8. Cable in particular a USB-OTG, USB-C, Lightning or Thunderbolt cable, comprising a further connector, in particular a USB type-A, Lightning or Thunderbolt connector; and the communication adapter of claim 1.

9. Connector, in particular a USB-OTG, USB-C, Lightning or Thunderbolt connector, comprising the communication adapter of claim 1, wherein the connector comprises a first antenna connection and a second antenna connection.

10. Communication adapter system comprising the connector of claim 9 and an adhesive film attachable to the mobile device, wherein a first MICS-band antenna and a second MICS-band antenna are arranged on a surface of the adhesive film.

11. Computer implemented method for transferring data between an implantable medical device, in particular a pacemaker, a defibrillator, a cardiac monitor and/or a neuro-stimulator, and a mobile device, in particular a smartphone or tablet computer, by means of a communication adapter, comprising the steps of:
providing a MICS telemetry interface for data transfer between the communication adapter and the implantable medical device;
providing a communication and power supply interface, in particular a USB-OTG, USB-C, Lightning or Thunderbolt interface, for data transfer between the communication adapter and the mobile device and for power supply from the mobile device to the communication adapter, wherein the communication adapter transfers data sent via the MICS telemetry interface by the implantable medical device to the mobile device via the communication and power supply interface and transfers data sent via the communication and power supply interface by the mobile device to the implantable medical device via the MICS telemetry interface.

12. Computer program with program code to perform the method of claim 11 when the computer program is executed on a computer.

13. Computer-readable data carrier containing program code of a computer program for performing the method of claim 11 when the computer program is executed on a computer.
